(19) United States
(12) Patent Application Publication
(10) Pub. No.: US 2010/0030157 A1
(43) Pub. Date: Feb. 4, 2010

(54) Drug Retention Cap

(76) Inventors: Cesario Dos Santos, Aliso Viejo, CA (US); Robert J. Sanchez, JR., Oceanside, CA (US)

Correspondence Address: ALCON, IP LEGAL, TB4-8, 6201 SOUTH FREEWAY, FORT WORTH, TX, 76134, US

(21) Appl. No.: 12/364,004

(22) Filed: Feb. 2, 2009

Related U.S. Application Data
(60) Provisional application No. 61/026,235, filed on Feb. 5, 2008.

(51) Int. Cl. A61M 5/31; A61B 19/02

(52) U.S. Cl. 604/199; 604/403

(57) ABSTRACT

A drug retention cap has a cap section and a drug retention pin. The cap section has a generally dome-shaped top end and a cylindrical portion extending downward from the generally dome-shaped top end. The top end of the cap section is closed, and the bottom end is open. The open bottom end is configured to receive a needle of a drug delivery device. The drug retention pin extends from near the apex of the dome-shaped top end downward toward the open bottom end. The drug retention pin is configured to fit inside a hollow needle.

This application claims the benefit of U.S. 61/026,236, filed Feb. 5, 2008.

### BACKGROUND OF THE INVENTION

A preloaded drug delivery device, like a manual or automated syringe, may have a pharmaceutical preloaded at a pharmaceutical manufacturing facility. In such a case, a precise dosage is preloaded into the device at the facility, the device is packaged and shipped, and the end-user is able to activate the device to deliver the precise dosage.

In many cases, it is desirable to preload a drug in a drug delivery device at a centralized manufacturing or processing facility. Preloading a device in such an environment can lead to very precise dosing—more precise than is attainable by a typical health professional in an office setting. In addition, some pharmaceuticals require special handling or must be kept under certain environmental conditions.

For example, a pharmaceutical suspended in or mixed with a phase transition compound requires special handling. One typical class of phase transition compounds are solid or semi-solid at room temperature and less viscous at higher temperatures. Such compounds behave like lipids or waxes. Such compounds can be extremely useful in ophthalmic injections, where a given compound/drug mixture can be heated to a less viscous state and injected into the eye to form a bolus that erodes over time.

It would be extremely difficult for the typical health professional to load such compounds into a syringe. Such compounds must be heated to a proper temperature and placed in a drug delivery device or subcomponent thereof. This difficulty in dosing requires that the phase transition compound/drug mixture be loaded into a specialized drug delivery device at a manufacturing or processing facility.

After the drug delivery device is preloaded and packaged, it may be subjected to various stresses during shipment. For example, the device may be subjected to extremes in temperature or physical stresses. In addition, the device may be improperly stored in an office setting or may experience stresses similar to those experienced during shipment. Under such conditions, the precise dosage that has been preloaded in the device may become dislodged or displaced from the device. What is needed is a protective cap to preserve the integrity of a preloaded dosage.

### SUMMARY OF THE INVENTION

In one embodiment consistent with the principles of the present invention, the present invention is a drug retention cap that has a cap section and a drug retention pin. The cap section has a generally dome-shaped top end and a cylindrical portion extending downward from the generally dome-shaped top end. The top end of the cap section is closed, and the bottom end is open. The open bottom end is configured to receive a needle of a drug delivery device. The drug retention pin extends from near the apex of the dome-shaped top end downward toward the open bottom end. The drug retention pin is configured to fit inside a hollow needle.

In another embodiment consistent with the principles of the present invention, the present invention is a drug retention cap that has a cap section and a drug retention pin. The cap section has a closed top end and a side wall portion extending downward from the closed top end. The cap section has an open bottom end defined by a periphery of the side wall portion. The open bottom end is configured to receive a needle of a drug delivery device. The drug retention pin extends from the top end downward toward the open bottom end. The drug retention pin is configured to fit inside a hollow needle.

In another embodiment consistent with the principles of the present invention, the present invention is a transportable drug delivery assembly including a drug delivery device and a drug retention cap. The drug delivery device includes a drug chamber configured to hold a quantity of a pharmaceutical, a plunger slidably disposed in the drug chamber, a needle fluidly coupled to the drug chamber and extending from the drug chamber, and a housing at least partially enclosing the drug chamber. The drug retention cap has a cap section and a drug retention pin. The cap section has a generally dome-shaped top end and a cylindrical portion extending from the generally dome-shaped top end. The top end of the cap section is closed, and the bottom end is open. The open bottom end is configured to receive the needle of the drug delivery device. The drug retention pin extends from near the apex of the dome-shaped top end downward toward the open bottom end. The drug retention pin is configured to fit inside the needle.

In another embodiment consistent with the principles of the present invention, the present invention is a transportable drug delivery assembly including a drug delivery device and a drug retention cap. The drug delivery device has a drug chamber configured to hold a quantity of a pharmaceutical, a plunger slidably disposed in the drug chamber, a needle fluidly coupled to the drug chamber and extending from the drug chamber, and a housing at least partially enclosing the drug chamber. The drug retention cap has a cap section and a drug retention pin. The cap section has a closed top end and a side wall portion extending from the top end. The cap section also has an open bottom end defined by a periphery of the side wall portion. The open bottom end is configured to receive the needle of the drug delivery device. The drug retention pin extends from the top end downward toward the open bottom end. The drug retention pin is configured to fit inside the needle.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1A is a cross section view of a drug retention cap and a portion of a drug delivery device.

FIG. 1B is a cross section view of a drug retention cap fitted onto a portion of a drug delivery device.

### DETAILED DESCRIPTION OF THE PREFERRED EMBODIMENTS

Reference is now made in detail to the exemplary embodiments of the invention, examples of which are illustrated in the accompanying drawings. Wherever possible, the same reference numbers are used throughout the drawings to refer to the same or like parts.

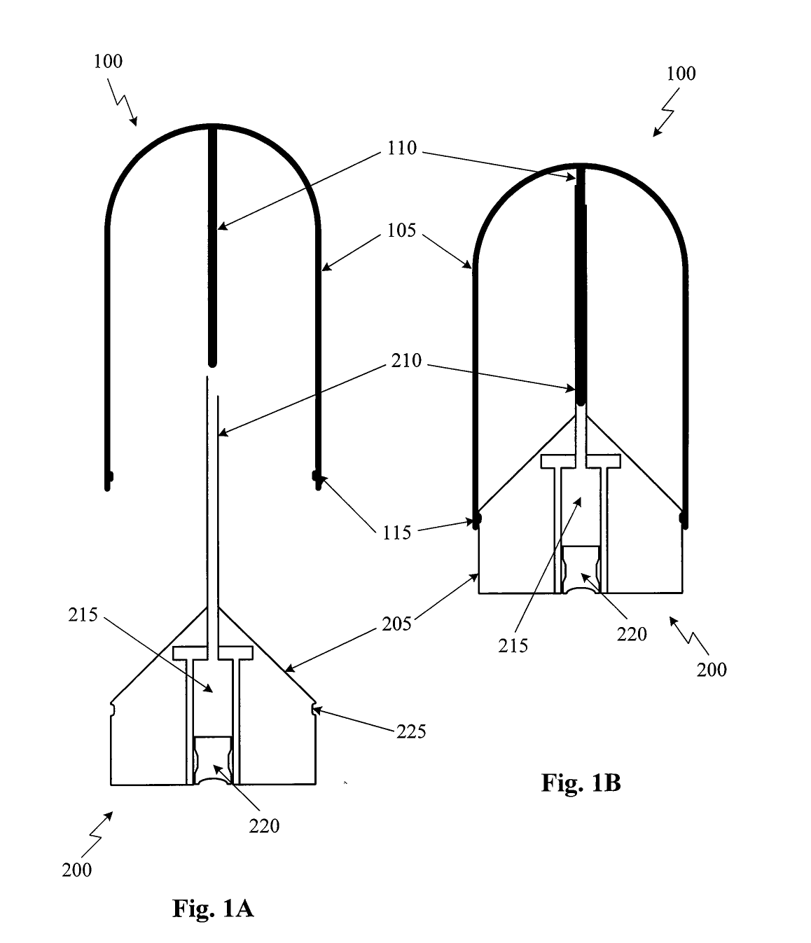

FIG. 1A is cross section view of a drug retention cap and a portion of a drug delivery device according to the principles of the present invention. FIG. 1B is cross section view of a drug retention cap fitted onto a portion of a drug delivery device according to the principles of the present invention. A drug retention cap 100 can be fitted over a drug delivery device 200 to retain a pharmaceutical in the device.

Drug retention cap 100 includes outer shell 105, drug retention pin 110 and optional tabs 115. In the embodiment pictured, outer shell 105 is shaped like a silo. As such, a generally cylindrical base is topped by a dome. Outer shell 105 has a drug retention pin 110 that extends from at or near the apex of the dome downward toward the cap opening. Drug retention pin 110 is shaped to fit inside a needle, such as needle 210, and its length is such that it extends into the hollow bore of needle 210. Drug retention pin 110 may extend to any depth in needle 210. Optional tabs 115 are provided to secure drug retention cap 100 to a drug delivery device 200.

Drug retention cap 100 is preferably made of a polymeric or plastic material and is molded as a single piece. Drug retention pin 110 is made of or coated with a material that is approved for contact with a pharmaceutical.

A portion of a drug delivery device 200 generally includes a needle 210, a drug chamber 215, a plunger 220, a housing 205, and optional detents 225. Needle 210 is preferable a small gauge needle that is suitable for injection into the eye. For such ophthalmic applications, it is preferable to employ a small gauge needle to reduce trauma to the eye and provide for a sutureless injection. In such a case, a needle smaller than or equal to 23 gauge is preferred, with a 25 gauge needle being most preferable.

Drug chamber 215 holds the drug. While the drug chamber is typically cylindrical, it may be of any suitable shape and size. In this case, a mixture of a phase transition compound and a pharmaceutical is contained in drug chamber 215. A phase transition compound is in a solid or semi-solid state at lower temperatures and in a more liquid state at higher temperatures. Such a substance can be heated to a more liquid state and injected into an eye where it forms a bolus that erodes over time. Likewise, a reverse gelation compound may be used. A reverse gelation compound is in a solid or semi-solid state at higher temperatures and in a more liquid state at lower temperatures. Such a compound can be cooled and injected into the eye where it forms a bolus that erodes over time. After being delivered into the eye, a phase transition compound or reverse gelation compound (which is solid or semi-solid at body temperature) erodes over time providing a quantity of drug over an extended period of time. Using a phase transition compound or reverse gelation compound provides better drug dosage with fewer unpleasant injections.

Plunger 220 is adapted to slide in drug chamber 215. When plunger 220 is driven upward toward needle 210, the drug in drug chamber 215 is expelled through needle 210. Plunger 220 is driven by a shaft (not shown) which may be actuated manually or automatically, for example, by a linear actuator (not shown). Plunger 220 is typically made of rubber or a polymeric material.

Housing 205 forms an outer skin on the portion of drug delivery device 200 depicted. Housing 205 is typically made of an impervious material such as a plastic. Optional detents 225 on housing 205 receive optional tabs 115 to hold drug retention cap 100 in place.

FIG. 1B shows a drug retention cap 100 placed on a portion of a drug delivery device 200. In this configuration, the unit is ready for shipment. A drug, such as a phase transition/pharmaceutical mixture, is preloaded in drug chamber 215. Plunger 220 is seated in place (or the drug may be loaded into drug chamber 215 with plunger 220 in place). Drug retention cap 100 is placed over the needle end of the device such that drug retention pin 110 fits into the hollow bore of needle 210. In this manner, drug retention pin 110 acts as a plug that retains the drug in drug chamber 215.

From the above, it may be appreciated that the present invention provides an improved apparatus for transporting a drug delivery device. A drug retention cap is placed over a drug delivery device to retain the drug in the device. A drug retention pin acts as a plug that holds the drug in place. The present invention is illustrated herein by example, and various modifications may be made by a person of ordinary skill in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A drug retention cap comprising:
a cap section with a generally dome-shaped top end and a cylindrical portion extending from the generally dome-shaped top end, the cap section having a closed top end and an open bottom end, the open bottom end for receiving a needle of a drug delivery device; and
a drug retention pin extending from near the apex of the dome-shaped top end downward toward the open bottom end, the drug retention pin configured to fit inside a hollow needle.

2. The drug retention cap of claim 1 further comprising:
one or more tabs, the one or more tabs configured to engage one or more detents on a drug delivery device to secure the drug retention cap to the drug delivery device.

3. The drug retention cap of claim 1 wherein the cap section is made of a polymer.

4. The drug retention cap of claim 1 wherein the drug retention pin is made of or coated with a material that is approved for contact with a pharmaceutical.

5. A drug retention cap comprising:
a cap section with a closed top end and a side wall portion extending from the top end, the cap section having an open bottom end defined by a periphery of the side wall portion, the open bottom end for receiving a needle of a drug delivery device; and
a drug retention pin extending from the top end downward toward the open bottom end, the drug retention pin configured to fit inside a hollow needle.

6. The drug retention cap of claim 5 further comprising:
one or more tabs, the one or more tabs configured to engage one or more detents on a drug delivery device to secure the drug retention cap to the drug delivery device.

7. A transportable drug delivery assembly comprising:
a drug delivery device comprising a drug chamber configured to hold a quantity of a pharmaceutical, a plunger slidably disposed in the drug chamber, a needle fluidly coupled to the drug chamber and extending from the drug chamber, and a housing at least partially enclosing the drug chamber; and
a drug retention cap comprising a cap section with a generally dome-shaped top end and a cylindrical portion extending from the generally dome-shaped top end, the cap section having a closed top end and an open bottom end, the open bottom end for receiving the needle of the drug delivery device; and a drug retention pin extending from near the apex of the dome-shaped top end downward toward the open bottom end, the drug retention pin configured to fit inside the needle.

8. The transportable drug delivery assembly of claim 7 further comprising:
one or more tabs located on the drug retention cap, the one or more tabs configured to engage one or more detents located on the drug delivery device to secure the drug retention cap to the drug delivery device.

9. The transportable drug delivery assembly of claim 7 wherein the drug retention cap is made of a polymer.

10. The transportable drug delivery assembly of claim 7 wherein the drug retention pin is made of or coated with a material that is approved for contact with a pharmaceutical.

11. The transportable drug delivery assembly of claim 7 wherein the needle is smaller than or equal to 23 gauge and the drug retention pin is configured to fit inside the needle and to act as a plug that retains a pharmaceutical in the drug chamber.

12. A transportable drug delivery assembly comprising:
a drug delivery device comprising a drug chamber configured to hold a quantity of a pharmaceutical, a plunger slidably disposed in the drug chamber, a needle fluidly coupled to the drug chamber and extending from the drug chamber, and a housing at least partially enclosing the drug chamber; and
a drug retention cap comprising a cap section with a closed top end and a side wall portion extending from the top end, the cap section having an open bottom end defined by a periphery of the side wall portion, the open bottom end for receiving the needle of the drug delivery device; and a drug retention pin extending from the top end downward toward the open bottom end, the drug retention pin configured to fit inside the needle.

13. The transportable drug delivery assembly of claim 12 further comprising:
one or more tabs located on the drug retention cap, the one or more tabs configured to engage one or more detents located on the drug delivery device to secure the drug retention cap to the drug delivery device.

14. The transportable drug delivery assembly of claim 12 wherein the drug retention cap is made of a polymer.

15. The transportable drug delivery assembly of claim 12 wherein the drug retention pin is made of or coated with a material that is approved for contact with a pharmaceutical.

16. The transportable drug delivery assembly of claim 12 wherein the needle is smaller than or equal to 23 gauge, and the drug retention pin is configured to fit inside the needle and to act as a plug that retains a pharmaceutical in the drug chamber.
